(19) United States
(12) Patent Application Publication
(10) Pub. No.: US 2024/0350659 A1
(43) Pub. Date: Oct. 24, 2024

(54) COMPOSITIONS AND METHODS FOR BIMODAL ANTI-VIRAL COMBINATION THERAPY

(71) Applicant: Haus Bioceuticals, Inc., Oklahoma City, OK (US)

(72) Inventors: Michael Centola, Oklahoma City, OK (US); Philip ALEX, Oklahoma City, OK (US)

(21) Appl. No.: 18/684,269

(22) PCT Filed: Aug. 19, 2022
(86) PCT No.: PCT/US22/75216

§ 371 Date: Feb. 16, 2024

Related U.S. Application Data
(60) Provisional application No. 63/234,902, filed on Aug. 19, 2021.

(51) Int. Cl. A61K 47/69; A61K 31/365; A61K 31/7048; A61P 31/14

(52) U.S. Cl. CPC A61K 47/6951; A61K 31/365; A61K 31/7048; A61P 31/14

(57) ABSTRACT

Pharmaceutical compositions, kits, and methods are disclosed for use in treating or reducing the occurrence of a viral infection in a patient. The compositions, kits, and methods utilize at least one anti-Pathogenic Host Response (anti-PHR) agent, either alone or in combination with at least one anti-viral agent.

### CROSS REFERENCE TO RELATED APPLICATIONS/INCORPORATION BY REFERENCE

The subject application claims benefit under 35 USC § 119 (e) of U.S. Ser. No. 63/234,902, filed Aug. 19, 2021. The entire contents of the above-referenced patent application(s) are hereby expressly incorporated herein by reference.

### STATEMENT REGARDING FEDERALLY SPONSORED RESEARCH OR DEVELOPMENT Not Applicable.

### BACKGROUND

Newly identified viruses, such as coronaviruses, can be difficult to treat because they are not sufficiently characterized. Severe acute respiratory syndrome coronavirus 2 (SARS-COV-2) is a newly-emergent coronavirus which causes a severe acute respiratory disease, COVID-19 (Coronavirus Disease 2019). Fever, cough, and fatigue are the most commonly reported symptoms of COVID-19. Pneumonia with shortness of breath is the most serious manifestation of the infection, and the disease can cause respiratory failure resulting in death. The virus spreads readily from person to person primarily through infected secretions, such as saliva and respiratory droplets or aerosols. Evidence supports spread by both symptomatic and asymptomatic individuals.

About 5% of COVID-19 patients experience complications including septic shock, acute respiratory distress syndrome (ARDS), acute cardiac or kidney injury, and thromboembolic events, including disseminated intravascular coagulation (DIC) and pulmonary embolism (PE). These complications are thought to be manifestations of the cytokine storm triggered by the host immune response to the virus (see FIG. 1). In critically ill patients, ARDS was the most common complication in 67% of the patients, with a 28-day mortality of 61.5%. DIC has been widely reported in COVID-19. PE in COVID-19 patients has been reported in a few studies. A recent study pointed to a higher incidence of PE with 23% in severe COVID-19 patients.

Many currently available anti-viral agents are being studied to determine their effects on SARS-COV-2 infection. However, only limited efficacy of approved and experimental anti-viral drugs has been observed. Thus far, no therapeutic agents have been identified that prevent or treat SARS-COV-2 infection.

For example, four repurposed anti-viral drugs (remdesivir, hydroxychloroquine, lopinavir, and interferon beta-1a) were each found to have little or no effect on hospitalized patients with Covid-19 in the WHO Solidarity Trial (WHO Solidarity Trial Consortium; N Engl J Med (published online Dec. 2, 2020) 384:497-511; DOI: 10.1056/NEJMoa2023184).

Also, the effects of ivermectin on the duration of symptoms in adults with mild COVID-19 was recently studied in a randomized clinical trial that included 476 patients. However, the duration of symptoms was not significantly different for patients who received a 5-day course of ivermectin compared with placebo; as such, these findings do not support the use of ivermectin for treatment of mild COVID-19 (López-Medina et al.; JAMA (published online Mar. 4, 2021) doi: 10.1001/jama.2021.3071).

In addition, there are limits on various treatment modalities because of the toxic and lethal effects of the Pathogenic Host Response (PHR), a hyperinflammatory response profile that has been shown to be the predominant cause of patient mortality in COVID-19. There is evidence of direct invasion of endothelial cells by the SARS-COV-2 virus, leading to cell injury and microvascular inflammation, which plays a central role in the pathogenesis of the acute respiratory distress syndrome (ARDS) mentioned above. This is akin to an autoinflammatory disease or septic shock, subsequently causing multisystem organ failure. Inflammation, fibrosis, endothelial damage, vascular leakage, coagulation, and alveolar wall thickening have all observed in COVID-19 patients. Therefore, compositions and methods for modulating PHR are desperately needed.

In view of the continuing threat to human health, there is an urgent need for preventive and therapeutic antiviral therapies for SARS-COV-2. It is to such compositions and methods that the present disclosure is directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1 graphically illustrates the key events in the pathophysiological progression of COVID-19 and demonstrates how PHR is the primary cause of morbidity and mortality in COVID-19 infections due to inflammation-induced lung injury and organ failure.

FIG. 2 graphically depicts one non-limiting embodiment of combinatorial therapy constructed in accordance with the present disclosure and comprising at least one anti-PHR component and at least one anti-viral component.

FIG. 3 graphically depicts the survival rate observed in a human ACE2 transgenic COVID-19 mouse model utilizing one non-limiting embodiment of an anti-PHR agent constructed in accordance with the present disclosure. All treatments were administered daily, starting at Day 0 pre-infection.

FIG. 4 graphically depicts the effect of the anti-PHR agent utilized in FIG. 3 on body weight. All treatments were administered daily, starting at Day 0 pre-infection.

FIG. 5 graphically depicts the effect of the anti-PHR agent utilized in FIG. 3 on SARS-CoV-2 viral titer. All treatments were administered daily, starting at Day 0 pre-infection.

FIG. 6 graphically depicts the survival rate observed in a human ACE2 transgenic COVID-19 mouse model utilizing various non-limiting embodiments of combinatorial therapies constructed in accordance with the present disclosure. All treatments were administered daily, starting at Day 1 post infection.

FIG. 7 graphically depicts the effect of the various combinatorial therapies utilized in FIG. 6 on body weight. All treatments were administered daily, starting at Day 1 post infection.

FIG. 8 graphically depicts the effects of the individual components of the combinatorial therapy utilized in FIG. 6 on body weight. β-cyclodextrin Rutin was administered daily, starting at Day 2 post infection; Ivermectin was administered once on Day 2 post infection.

FIG. 9 graphically depicts the effects of the individual components of the combinatorial therapy utilized in FIG. 6 on SARS-COV-2 viral titer. β-cyclodextrin Rutin was administered daily, starting at Day 2 post infection; Ivermectin was administered once on Day 2 post infection.

FIG. 10 graphically depicts a comparison of the survival rate observed in a human ACE2 transgenic COVID-19 mouse model utilizing one non-limiting embodiment of a combinatorial therapy in accordance with the present disclosure or its individual components. All treatments were administered daily, starting at Day 2 post infection.

FIG. 11 graphically depicts a comparison of the body weight observed utilizing the combinatorial therapy or its individual components of FIG. 10. All treatments were administered daily, starting at Day 2 post infection.

FIG. 12 graphically depicts a comparison of the body weight observed in a human ACE2 transgenic COVID-19 mouse model utilizing native anti-PHR agents or high bioavailability anti-PHR agents constructed in accordance with the present disclosure. All treatments were administered daily, starting at Day 2 post infection.

FIG. 13 graphically depicts a comparison of survival rate observed utilizing native anti-PHR agents or high bioavailability anti-PHR agents. All treatments were administered daily, starting at Day 2 post infection.

FIG. 14 contains a flow chart for patient disposition of a two-week, comparator-based, pilot study to evaluate the safety and efficacy of a combinatorial therapy constructed in accordance with the present disclosure compared to standard of care in 40 patients with COVID-19.

FIG. 15 graphically depicts the proportion of patients with a negative SARS-COV-2 PCR result after treatment with combinatorial therapy or its individual components.

FIG. 16 graphically depicts the ability of the anti-PHR agent rutin and cyclodextrin-modified forms thereof to inhibit SARS-COV-2 replication in vitro. “UNRX”—Untreated. “1:1 BCDRUT”—Betacyclodextrin Rutin, produced with a 1:1 molar ratio of betacyclodetrin to rutin; cells treated at 1 μM final concentration. “1:1 HPGCDRUT”—Hydroxypropylgammacyclodextrin Rutin produced with a 1:1 molar ratio of hydroxypropylgammacyclodextrin to rutin; cells treated at 1 μM final concentration. “1:1 GCDRUT”—Gammacyclodextrin Rutin produced with a 1:1 molar ratio of gammacyclodextrin to rutin; cells treated at 1 μM final concentration. “1:1 ACDRUT”—Alphacyclodextrin Rutin produced with a 1:1 molar ratio of alphacyclodextrin to rutin; cells treated at 1 μM final concentration. “RUT”—Rutin, no cyclodextrin; cells treated at 1 μM final concentration.

FIG. 17 graphically depicts the ability of the anti-PHR agent rutin and cyclodextrin-modified forms thereof to inhibit SARS-COV-2 replication in vitro. “2:1 BCDRUT”—Betacyclodextrin Rutin produced with a 2:1 molar ratio of betacyclodetrin to rutin. “1:1 BCDRUT”—Betacyclodextrin Rutin produced with a 1:1 molar ratio of betacyclodetrin to rutin. “2:1 BCDRUT”—Hydroxypropylbetacyclodextrin Rutin produced with a 2:1 molar ratio of Hydroxypropylbetacyclodetrin to rutin. “1:1 BCDRUT”—Hydroxypropylbetacyclodextrin Rutin produced with a 1:1 molar ratio of Hydroxypropylbetacyclodetrin to rutin. “RUT”—Rutin, no cyclodextrin; 1, 10, and 100 correspond to 1, 10, and 100 mg/ml treatment concentrations, respectively.

FIG. 18 graphically depicts the ability of various formulations containing a combination of anti-PHR agents to inhibit SARS-COV-2 replication in vitro. The entire formulation containing a combination of various anti-PHR agents is labeled as “All Supplement” or “APHR Combination 1” and includes Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT). The remaining formulations, labeled SUP2-SUP9, include all of the agents present in the APHR Combination 1 with the exception of one agent. That is, SUP2 contains all of the APHR Combination 1 except BCD-quercetin, SUP3 contains all of the APHR Combination 1 except hesperidin, etc.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, assemblies, and/or methods disclosed herein can be made and executed without undue experimentation in light of the present disclosure. While the compositions, kits, assemblies, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically acceptable excipient” refers to any carrier, vehicle, and/or diluent known in the art or otherwise contemplated herein that may improve solubility, deliverability, dispersion, stability, and/or conformational integrity of the compositions disclosed herein.

The terms “patient” and “subject” are used herein interchangeably and will be understood to include human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes. The term “treatment” includes a detectable or measurable improvement in a subject's condition and/or at least one symptom thereof.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one infection or condition. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

In addition, an “effective amount” of an active agent of the present disclosure refers to an amount which is effective in controlling, reducing, or inhibiting a condition as described herein, such as (but not limited to) a viral infection and/or the effects associated therewith. The term “controlling” is intended to refer to all processes wherein there may be a slowing, interrupting, arresting, or stopping of the progression of the condition and does not necessarily indicate a total elimination of the symptoms of the condition.

The term “effective amount” is further meant to define an amount resulting in the improvement of any parameters or clinical symptoms characteristic of a condition. The actual dose will vary with the patient's overall condition, the seriousness of the condition or symptoms, and contraindications. As used herein, the term “effective amount” also means the total amount of each active agent (component) of the pharmaceutical composition or method that is sufficient to show a meaningful patient benefit, i.e., reduction of a condition. When applied to an individual active ingredient, administered alone, the term refers to that ingredient alone. When applied to a combination, the term refers to combined amounts of the active agent(s) that results in the therapeutic effect, whether administered in combination, serially or simultaneously.

The term “ameliorate” means a detectable or measurable improvement in a subject's condition or symptom thereof. A detectable or measurable improvement includes a subjective or objective decrease, reduction, inhibition, suppression, limit, or control in the occurrence, frequency, severity, progression, or duration of the condition, or an improvement in a symptom or an underlying cause or a consequence of the condition, or a reversal of the condition. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most, or all adverse symptoms, complications, consequences, or underlying causes associated with the condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition (e.g., stabilizing), over a short or long duration of time (e.g., seconds, minutes, hours).

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), the treatment of viral infections (such as, but not limited to, COVID-19) using a bimodal combination therapy are envisioned, along with compositions for use in said treatments, kits containing same, and methods of producing these compositions.

Several avenues are currently being pursued to find treatments for COVID-19, and several therapeutics (such as, but not limited to, virucides, anti-malarial, and anti-inflammatory drugs) and vaccine solutions are being investigated. However, an effective treatment for COVID-19 has not yet been elucidated.

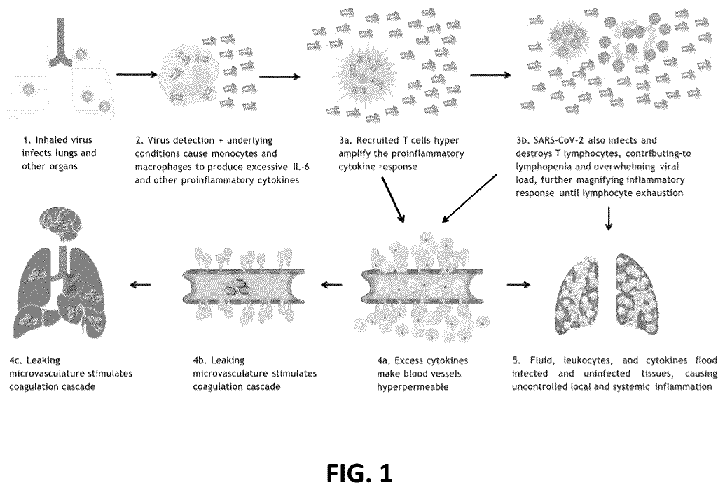

In addition, there are limits on various treatment modalities because of the toxic and lethal effects of the Pathogenic Host Response (PHR). For example, the acute respiratory distress syndrome (ARDS) observed in some COVID-19 patients is akin to an autoinflammatory disease or septic shock. Inflammation, fibrosis, endothelial damage, vascular leakage, coagulation, and alveolar wall thickening have all observed in COVID-19 patients. FIG. 1 illustrates how PHR is the primary cause of morbidity and mortality for COVID-19 patients due to inflammation-induced lung injury and organ failure.

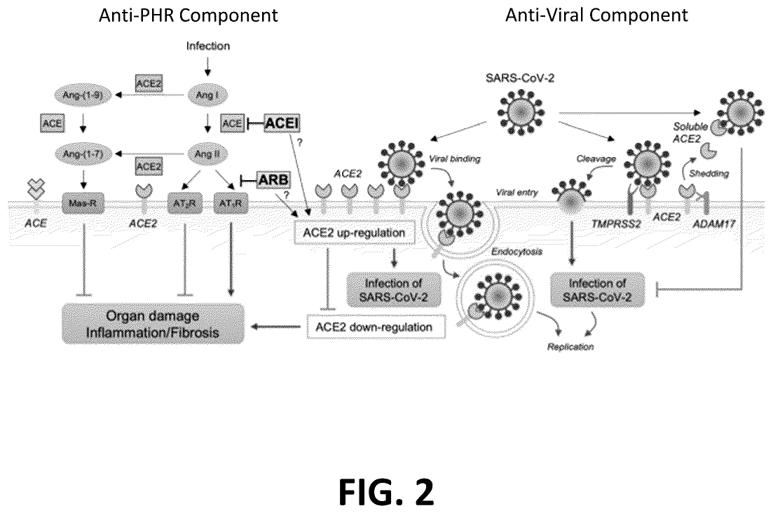

The compositions and therapy methods of the present disclosure include a bimodal combinatorial therapy involves the combination of at least one anti-PHR component with at least one anti-viral component (FIG. 2). This bimodal combinatorial therapy has been demonstrated to match the anti-viral activity of the standard of care while being more effective at alleviating the signs and symptoms of COVID-19 infection.

Certain non-limiting embodiments of the present disclosure are directed to a method of treating or reducing the occurrence of a viral infection in a patient. The method comprises the steps of: (1) administering at least one dose of at least one anti-viral agent to the patient; and (2) administering at least one dose of at least one anti-Pathogenic Host Response (anti-PHR) agent to the patient. The at least one anti-PHR agent is administered simultaneously or wholly or partially sequentially with the at least one anti-viral agent.

The term “anti-Pathogenic Host Response agent” or “anti-PHR agent” refers to any compound that is capable of preventing or reducing the Pathogenic Host Response to a viral infection in a patient. These agents modulate key aspects of virally-induced inflammatory cascades, including, but not limited to, inflammatory signaling (e.g., cytokine storms), fibrosis, and coagulation, which mediate tissue and organ destruction and thereby increase morbidity and mortality.

The methods of the present disclosure may be utilized to treat or reduce the occurrence of viral infections caused by any viruses known in the art or otherwise contemplated herein. Non-limiting examples of viruses that can be treated in accordance with the present disclosure include adenoviruses, astroviruses, coronaviruses (such as, but not limited to, severe acute respiratory syndrome coronavirus (SARS-COV) or Middle East respiratory syndrome coronavirus (MERS-COV)), Coxsackie viruses, cytomegaloviruses (CMV), Ebola viruses, echoviruses, encephalitis viruses, enteroviruses, Epstein-Barr viruses (EBV), erythroviruses, hantaviruses, hepatitis viruses, herpes viruses (HSV), human immunodeficiency viruses (HIV), influenza viruses, noroviruses, papilloma viruses, parainfluenza viruses, paramyxoviruses, polio viruses, rabies viruses, respiratory syncytial viruses (RSV), rhinoviruses, rotoviruses, rubella viruses, rubeola viruses, Swine flu (H1N1) viruses, Varicella-Zoster viruses, West Nile viruses, Zika viruses, hemorrhagic fever viruses, yellow fever viruses, dengue viruses, and the like, as well as variants thereof.

For example, but not by way of limitation, the compositions and methods may be utilized to treat a coronavirus infection. Non-limiting examples of coronaviruses that cause infections that can be treated in accordance with the present disclosure include severe acute respiratory syndrome coronavirus (SARS-COV), severe acute respiratory syndrome coronavirus 2 (SARS-COV-2, the virus that causes COVID-19), Middle East respiratory syndrome coronavirus (MERS-COV), human coronavirus 229E (HCOV-229E), human coronavirus OC43 (HCoV-OC43), human coronavirus NL63 (HCoV-NL63), and human coronavirus HKU1 (HCoV-HKU1), as well as variants thereof.

In a particular (but non-limiting) embodiment, the compositions and methods of the present disclosure are utilized to treat infection with SARS-COV-2 or a variant thereof.

Any anti-viral agents known in the art or otherwise contemplated herein that can be utilized to treat one or more of the viral infections described herein above may be utilized in the compositions and methods of the present disclosure. Non-limiting examples of anti-viral agents that may be utilized in accordance with the present disclosure include ivermectin, remdesivir, atorvastatin, montelukast, hydroxychloroquine, chloroquine, catechin, curcumin, quercetin, rutin, nafamostat mesylate, 6-mercaptopurine (6MP), zinc, lopinavir, ritonavir, oseltamivir, fexofenadine, ribavirin, favipiravir, licorice extract, andrographis extract, interferon-beta, dexamethasone, hydrocortisone, methylprednisolone, colchicine, convalescent plasma, ritonavir, molnupiravir, xocova, ensovibep, at least one SARS-COV-2 monoclonal antibody, Tocilizumab, Molnupiravir, vitamin D, zinc, and the like, as well as any combinations thereof (including, but not limited to, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or more of the above agents).

SARS-COV-2 monoclonal antibodies are known in the art and commercially available. Non-limiting examples thereof include Tixagevimab (Astra Zeneca, Cambridge, UK), Cilgavimab (Astra Zeneca), Bebtelovimab (Eli Lilly, Indianapolis, IN), Sotrovimab (GSK, Brentford, UK), Bamlanivimab (Eli Lilly), Etesevimab (Eli Lilly), Casirivimab (Regeneron, Tarrytown, NY), Imdevimab (Regeneron), as well as combinations thereof (such as, but not limited to, Tixagevimab/Cilgavimab, Bamlanivimab/Etesevimab, Casirivimab/Imdevimab, etc.). Therefore, no further description thereof is deemed necessary.

In particular (but non-limiting) embodiments, the anti-viral agent comprises ivermectin.

The anti-PHR agent may be utilized in its native or existing form, or the agent may be modified to increase the bioavailability and/or stability thereof. Any methods of increasing bioavailability/stability known in the art or otherwise contemplated herein may be utilized in accordance with the present disclosure. Non-limiting examples include binding/complexation with a cyclodextrin, lipid-based encapsulation (such as, but not limited to, emulsion/nanoemulsion, solid lipid nanoparticles, liposomes/nanoliposomes, etc.), phospholipid encapsulation, protein and amino acid encapsulation, binding/complexation to nanoparticles, use of adjuvants (such as, but not limited to, piperine and black pepper), and the like.

For example (but not by way of limitation), the anti-PHR agent may be modified by binding/complexation with a cyclodextrin to enhance the bioavailability and/or stability thereof. Non-limiting examples of cyclodextrins that may be utilized in accordance with the present disclosure include alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), hydroxypropyl gamma-cyclodextrin (HPGD), and the like.

In particular (but non-limiting) embodiments, the anti-PHR agent is selected from the group consisting of rutin, sodium rutin, curcumin, quercetin, hesperidin, baicalin, green tea extract, rose extract, betulinic acid, tannic acid, bisabolol, a modified form thereof (such as, but not limited to, a cyclodextrin (CD)-modified form thereof), and combinations thereof (including, but not limited to, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or more of the above agents and/or modified forms thereof).

In a particular (but non-limiting) embodiment, the anti-PHR agent comprises at least one of curcumin, quercetin, rutin, sodium rutin, ACD-curcumin, BCD-curcumin, HEBCD-curcumin, HPBCD-curcumin, SBCD-curcumin, GCD-curcumin, HPGD-curcumin, ACD-quercetin, BCD-quercetin, HEBCD-quercetin, HPBCD-quercetin, SBCD-quercetin, GCD-quercetin, HPGD-quercetin, ACD-rutin, BCD-rutin, HEBCD-rutin, HPBCD-rutin, SBCD-rutin, GCD-rutin, HPGD-rutin, or any combination thereof.

In a particular (but non-limiting) embodiment, the at least one anti-viral agent comprises ivermectin, and the at least one anti-PHR agent comprises at least one of rutin, sodium rutin, BCD-rutin, or HPBCD-rutin.

Each of the method steps of administering the anti-viral agent and the anti-PHR agent may be performed once or multiple times. For example (but not by way of limitation), the method may further include the step of: (3) administering at least one additional dose of the at least one anti-PHR agent to the patient. When step (3) is included, the step may be performed once or multiple times. In a particular (but non-limiting) embodiment, step (3) is repeated on a daily basis for a period in a range of from about 1 day to about 60 days.

In at least certain non-limiting embodiments, each of the anti-viral agent(s) and the anti-PHR agent(s) administered in the methods of the present disclosure are present in the same pharmaceutical composition or two different pharmaceutical compositions, in which they may be combined with a pharmaceutically acceptable carrier. For example (but not by way of limitation), the pharmaceutical composition may contain, in addition to the anti-viral agent(s) and/or anti-PHR agent(s), one or more of a diluent, an excipient, a filler, a salt, a buffer, a stabilizer, a solubilizer, a vehicle, and other materials well known in the art, as well as any combination thereof. Suitable carriers, vehicles, and other components for pharmaceutical formulations are described, for example, in Remington: The Science and Practice of Pharmacy, 23rd ed (2020).

In certain particular (but non-limiting) embodiments, the pharmaceutical compositions of the present disclosure include at least one of Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT).

In certain particular (but non-limiting) embodiments, the pharmaceutical compositions of the present disclosure include at least two, at least three, at least four, at least five, at least six, at least seven, at least eight, at least nine, at least 10, at least 11, at least 12, at least 13, at least 14, at least 15, at least 16, or all of Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT).

Where used herein, the term “pharmaceutically acceptable carrier” refers to any compound used in combination (e.g., in a composition or formulation) with the anti-viral agent(s) and/or anti-PHR agent(s) of the present disclosure, for example, for aiding in delivery of the anti-viral agent(s) and/or anti-PHR agent(s) to the subject to be treated. The term “pharmaceutically acceptable” means a non-toxic material that does not interfere with the effectiveness of the biological activity of the active agent(s). The characteristics of the carrier will depend on the route of administration.

For example, but not by way of limitation, the anti-viral agent(s) and/or anti-PHR agent(s) may be dissolved (separately or together) in a physiologically acceptable pharmaceutical carrier and administered as either a solution or a suspension. Non-limiting examples of suitable pharmaceutically acceptable carriers include water; saline; dextrose solutions; fructose solutions; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the anti-viral agent(s) and/or anti-PHR agent(s) (and pharmaceutically acceptable carrier(s), if present). Examples of additional secondary compounds that may be present include, but are not limited to, diluents, fillers, salts, buffers, preservatives, stabilizers, solubilizers, wetting agents, emulsifying agents, dispersing agents, and other materials well known in the art.

The anti-viral agent(s) and/or anti-PHR agent(s) may be present in the same or different pharmaceutical compositions at any concentration that allows the pharmaceutical composition(s) to function in accordance with the present disclosure; for example, but not by way of limitation, the anti-viral agent(s) and/or anti-PHR agent(s) may each be present at a concentration of about 0.0001 wt %, about 0.005 wt %, about 0.001 wt %, about 0.005 wt %, about 0.01 wt %, about 0.05 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, and about 2 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or higher. In addition, the anti-viral agent(s) and/or anti-PHR agent(s) may each be present at a concentration that falls within a range formed of two of the above values (i.e., a range of from about 0.001 wt % to about 75 wt %, a range of from about 0.05 wt % to about 35 wt %, etc.); also, the anti-viral agent(s) and/or anti-PHR agent(s) may be present at a concentration that falls within a range of two values, each of which falls between two values listed above (i.e., a range of from about 0.03 wt % to about 22 wt %; a range of from about 0.08 wt % to about 63 wt %; etc.).

In addition, in certain non-limiting embodiments, the anti-viral agent(s) and/or anti-PHR agent(s) may each be present in the same or different pharmaceutical compositions at a specific molar concentration. Non-limiting examples of molar concentrations that may be utilized in accordance with the present disclosure include about 0.0001 M, about 0.0005 M, about 0.001 M, about 0.005 M, about 0.01 M, about 0.05 M, about 0.1 M, about 0.2 M, about 0.3 M, about 0.4 M, about 0.5 M, about 0.6 M, about 0.7 M, about 0.8 M, about 0.9 M, about 1 M, about 2 M, about 3 M, about 4 M, about 5 M, or higher. In addition, the anti-viral agent(s) and/or anti-PHR agent(s) may each be present at a molar concentration that falls within a range formed of two of the above values (i.e., a range of from about 0.0001 M to about 1 M, a range of from about 0.001 M to about 0.1 M, etc.); also, the anti-viral agent(s) and/or anti-PHR agent(s) may each be present at a concentration that falls within a range of two values, each of which falls between two values listed above (i.e., a range of from about 0.007 M to about 0.86 M, etc.).

The amount of each of the ant-viral agent(s) and anti-PHR agent(s) present in the pharmaceutical composition(s) that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases, infections, and/or other conditions involved; the degree, involvement, and/or severity of the diseases, infections, and/or conditions; the response of the individual subject; the particular anti-viral agent(s) and/or anti-PHR agent(s) administered; the mode of administration; the dose regimen selected; the use of concomitant medication; and other relevant circumstances. A therapeutically effective amount of each of the anti-viral agent(s) and/or anti-PHR agent(s) of the present disclosure also refers to an amount of each of the anti-viral agent(s) and/or anti-PHR agent(s) which is effective in controlling, reducing, or ameliorating the condition/infection to be treated or may refer to the amount of the anti-viral agent(s) and/or anti-PHR agent(s) required to achieve a prophylactic effect for the purpose of preventing, controlling, reducing, or ameliorating the condition/infection to be treated.

For example, but not by way of limitation, the therapeutically effective amount of pharmaceutical composition(s) will generally contain sufficient anti-viral agent(s) and/or anti-PHR agent(s) to deliver each agent in a range of from about 0.01 μg/kg to about 10 g/kg (weight of anti-viral agent(s) and/or anti-PHR agent(s)/body weight of patient). For example, but not by way of limitation, the composition will deliver about 0.1 μg/kg to about 1 g/kg, and more particularly about 1 μg/kg to about 500 mg/kg of the anti-viral agent(s) and/or anti-PHR agent(s).

Exemplary, non-limiting therapeutically or prophylactically effective amounts of each of the anti-viral agent(s) and/or anti-PHR agent(s), based on the subject's body weight, include about 0.01 μg/kg, about 0.05 μg/kg, about 0.1 μg/kg, about 0.5 μg/kg, about 1 μg/kg, about 5 μg/kg, about 10 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 75 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1 g/kg, and higher, as well as a range formed from two of the above values (i.e., a range of from about 1 μg/kg to about 100 mg/kg of the subject's body weight, a range of from about 1 μg/kg to about 500 mg/kg of the subject's body weight, etc.), as well as a range formed from two values, each of which falls between two of the above values (i.e., a range of from about 7 μg/kg to about 575 mg/kg, a range of from about 2 μg/kg to about 325 mg/kg, etc.).

Each pharmaceutical composition is formulated to contain an effective amount of anti-viral agent(s) and/or anti-PHR agent(s), wherein the amount depends on the animal to be treated and the condition to be treated. In certain embodiments, each of the anti-viral agent(s) and/or anti-PHR agent(s) is administered at a dose ranging from about 0.001 mg to about 100 g, a dose ranging from about 0.01 mg to about 10 g, a dose ranging from about 0.1 mg to about 10 g, a dose ranging from about 1 mg to about 10 g, a dose ranging from about 1 mg to about 9 g, a dose ranging from about 1 mg to about 8 g, a dose ranging from about 1 mg to about 7 g, a dose ranging from about 1 mg to about 6 g, a dose ranging from about 1 mg to about 5 g, a dose ranging from about 10 mg to about 10 g, a dose ranging from about 50 mg to about 5 g, a dose ranging from about 50 mg to about 5 g, a dose ranging from about 50 mg to about 2 g, a dose ranging from about 0.05 μg to about 1.5 mg, a dose ranging from about 10 μg to about 1 mg, a dose ranging from about 30 μg to about 500 μg, a dose ranging from about 0.1 μg to about 200 mg, a dose ranging from about 0.1 μg to about 5 μg, a dose ranging from about 5 μg to about 10 μg, a dose ranging from about 10 μg to about 25 μg, a dose ranging from about 25 μg to about 50 μg, a dose ranging from about 50 μg to about 100 μg, a dose ranging from about 100 μg to about 500 μg, a dose ranging from about 500 μg to about 1 mg, or a dose ranging from about 1 mg to about 2 mg. The specific dosage level for any particular subject depends upon a variety of factors including the activity of the specific peptide, the age, body weight, general health, sex, diet, time of administration, route of administration, and rate of excretion, drug combination, and the severity of the particular disease undergoing therapy.

In certain particular (but non-limiting) embodiments of the present disclosure, the dose of step (1) (i.e., the anti-viral agent) is in a range of from about 1 μg/kg to about 100 mg/kg, and the dose of step (2) (i.e., the anti-PHR agent) is in a range of from about 1 μg/kg to about 500 mg/kg.

The dosage of an administered pharmaceutical composition for the subject will vary depending upon such factors as the patient's age, weight, height, sex, general medical condition, and previous medical history. In certain non-limiting embodiments, the recipient is provided with a dosage of each of the anti-viral agent(s) and/or anti-PHR agent(s) that is in the range of from about 1 mg to about 1000 mg as a single infusion or single or multiple injections, although a lower or higher dosage also may be administered. The dosage may be in the range of from about 25 mg to about 100 mg of the anti-viral agent(s) and/or anti-PHR agent(s) per square meter (m²) of body surface area for a typical adult, although a lower or higher dosage also may be administered. Examples of dosages that may be administered to a human subject further include, for example, in a range of from about 1 mg to about 500 mg, a range of from about 1 mg to about 70 mg, or a range of from about 1 mg to about 20 mg, although higher or lower doses may be used.

Dosages may be repeated as needed, for example (but not by way of limitation), once every 10 minutes, once every 30 minutes, once every hour, once every two hours, once every three hours, once every four hours, once every five hours once every six hours, once every eight hours, once every 12 hours, once a day, once per week, etc. It may also be given less frequently, such as every other week for several months, or more frequently, such as twice weekly, or by continuous infusion.

The anti-viral and anti-PHR compositions of the present disclosure may be administered via one or more routes of administration using one or more of a variety of methods known in the art. As will be appreciated by the skilled artisan, the route and/or mode of administration will vary depending upon the desired results. Selected routes of administration include (but are not limited to) intravenous, intramuscular, intradermal, intraperitoneal, subcutaneous, spinal, or other parenteral routes of administration, for example by injection or infusion. The anti-viral agent(s) and/or anti-PHR agent(s) can be delivered alone or as pharmaceutical compositions by any means known in the art, e.g., systemically, regionally, or locally; by intra-arterial, intrathecal (IT), intravenous (IV), parenteral, intra-pleural cavity, or local administration, as subcutaneous, intra-tracheal (e.g., by aerosol), or transmucosal (e.g., buccal, bladder, vaginal, uterine, rectal, nasal mucosa). Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, compositions can be administered via a non-parenteral route, such as a topical, epidermal, or mucosal route of administration, for example, intranasally, orally, vaginally, rectally, sublingually, or topically. In one embodiment, the composition(s) is administered by infusion. In another embodiment, the composition(s) is administered subcutaneously. In another embodiment, the composition(s) is administered orally. In another embodiment, the composition(s) is administered to the ear canal. In another embodiment, the composition(s) is administered transdermally. In another embodiment, the composition(s) is administered to the lungs with no penetration, partial penetration, or complete penetration of the lung tissues.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra.

The compositions can be administered by the oral or nasal respiratory route for local or systemic effect. Compositions in pharmaceutically acceptable solvents may be nebulized by use of inert gases. Nebulized solutions may be inhaled directly from the nebulizing device, or the nebulizing device may be attached to a face mask tent or intermittent positive pressure breathing machine. Solution, suspension, or powder compositions may also be administered orally or nasally from devices which deliver the formulation in an appropriate manner.

When the pharmaceutical composition is to be used as an injectable material, it can be formulated into a conventional injectable carrier. Non-limiting examples of suitable carriers include biocompatible and pharmaceutically acceptable phosphate buffered saline solutions, which are particularly isotonic.

In certain non-limiting embodiments, the anti-viral agent(s) and/or anti-PHR agent(s) is provided as a lyophilized product that is reconstituted, such as (but not limited to) for injection. For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The same route of administration may be utilized for both the anti-viral agent(s) and the anti-PHR agents. Alternatively, the anti-viral agent(s) may be administered by a different administration route than the anti-PHR agent(s).

Practice of the methods of the present disclosure may include administering to a subject a therapeutically effective amount of the pharmaceutical composition(s) (containing the anti-viral agent(s) and/or anti-PHR agent(s) in any suitable systemic and/or local formulation), in an amount effective to deliver the desired dosage. The dosage can be administered, for example, but not by way of limitation, continuously or intermittently. In addition, the dosage can be administered on a one-time basis or administered at multiple times (for example, but not by way of limitation, from one to five times per day, or once or twice per week). The pharmaceutical composition may be administered either alone or in combination with other therapies, in accordance with the inventive concepts disclosed herein.

Each of the pharmaceutical compositions of the present disclosure can be administered in a single dose treatment or in multiple dose treatments on a schedule and over a time period appropriate to the age, weight, and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, whether the composition is used for prophylactic or curative purposes, etc. For example, in certain non-limiting embodiments, the composition is administered once per day, twice per day, three times per day, four times per day, five times per day, six times per day, seven times per day, eight times per day, nine times per day, 10 times per day, 12 times per day, or more frequently, or the composition may be administered every other day, once per week, twice per week, three times per week, four times per week, five times per week, six times per week, or seven times per week. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, or more.

Certain non-limiting embodiments of the present disclosure are directed to a kit for treatment of a viral infection. The kit includes at least one of any of the anti-viral agents described or otherwise contemplated herein (alone or in the form of any of the pharmaceutical compositions described or otherwise contemplated herein) and at least one of any of the anti-PHR agents described or otherwise contemplated herein (alone or in the form of any of the pharmaceutical compositions described or otherwise contemplated herein).

In addition, the kit may further contain one or more other component(s) or reagent(s) for use in treatment of the viral infection in accordance with the present disclosure. For example (but not by way of limitation), the kit may further contain one or more additional component, devices, or reagents utilized in administration of the anti-viral and/or anti-PHR agents. The nature of these additional component(s)/device(s)/reagent(s) will depend upon various factors such as (but not limited to) the route(s) of administration, and identification thereof is well within the skill of one of ordinary skill in the art; therefore, no further description thereof is deemed necessary.

Also, the various components/reagents present in the kit may each be in separate containers/compartments, or various components/reagents can be combined in one or more containers/compartments, depending on the cross-reactivity and stability of the components/reagents. In addition, the kit may include a set of written instructions explaining how to use the kit. A kit of this nature can be used in any of the methods described or otherwise contemplated herein.

Certain non-limiting embodiments of the present disclosure are directed to any of the pharmaceutical compositions disclosed or otherwise contemplated herein, wherein the pharmaceutical composition comprises at least one of any of the anti-viral agents disclosed or otherwise contemplated herein, at least one of any of the anti-PHR agents disclosed or otherwise contemplated herein, and at least one pharmaceutically acceptable carrier in which the at least one anti-viral agent and at least one anti-PHR agent are disposed.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

Many currently available anti-viral agents are being studied to determine their effects on SARS-COV-2 infection. However, only limited efficacy of approved and experimental anti-viral drugs has been observed. Thus far, no therapeutic agents have been identified that prevent or treat SARS-COV-2 infection. For example, four repurposed anti-viral drugs (remdesivir, hydroxychloroquine, lopinavir, and interferon beta-1a) were each found to have little or no effect on disease progression in patients with severe COVID-19 infection and failed the critical WHO Solidarity Trial (WHO Solidarity Trial Consortium; N Engl J Med (published online Dec. 2, 2020) 384:497-511; DOI: 10.1056/NEJMoa2023184). Also, ivermectin was not found to have any significant effect on the duration of symptoms in adults with mild COVID-19 in a randomized clinical trial that included 476 patients; as such, the findings from this trial do not support the use of ivermectin for treatment of mild COVID-19 (López-Medina et al.; JAMA (published online Mar. 4, 2021) doi: 10.1001/jama.2021.3071).

In addition, there are limits on various treatment modalities because of the toxic and lethal effects of the Pathogenic Host Response (PHR). For example, the acute respiratory distress syndrome (ARDS) observed in some COVID-19 patients is akin to an autoinflammatory disease or septic shock. Inflammation, fibrosis, endothelial damage, vascular leakage, coagulation, and alveolar wall thickening have all observed in COVID-19 patients. FIG. 1 illustrates how PHR is the primary cause of morbidity and mortality for COVID-19 patients due to inflammation-induced lung injury and organ failure. Therefore, compositions and methods for modulating PHR are desperately needed.

The compositions and therapy methods of the present disclosure are effective, safe, and inexpensive and utilize anti-inflammatory agents that modulate PHR. Certain embodiments of the compositions of the present disclosure include a bimodal combination therapy that includes at least one PHR modulator and at least one anti-viral agent. This combinatorial therapy is demonstrated herein to be highly efficacious in vivo and provided promising results in a proof-of-concept clinical trial.

As shown in FIG. 2, the bimodal combinatorial therapy of the present disclosure involves the combination of at least one anti-PHR component with at least one anti-viral component. This bimodal combinatorial therapy has been demonstrated to match the anti-viral activity of the standard of care while being more effective at alleviating the signs and symptoms of COVID-19 infection.

Anti-PHR agents were identified and selected based on in silico protein-binding models and anti-SARS-COV-2 and anti-SARS-COV-2-like activity, anti-viral, anti-inflammatory, anti-fibrotic, anti-coagulant, and/or bradykinin inhibitory activities. Then the candidates were prioritized based on mechanism of action (MOA), clinical potential, and safety, as well as meeting regulatory, cost, and supply chain constraints. Broad-based in vitro and in vivo screenings were then conducted to assess MOA and efficacy. The anti-PHR agents selected have anti-inflammatory, anti-coagulant, anti-lung fibrotic, and/or anti-viral activity, ameliorate acute lung injury, in silico SARS-COV-2 protein binding, and are designated as GRAS (Generally Regarded As Safe). In addition, the anti-PHR agents can be produced at high volume and scalable, and are low cost. The anti-PHR agents have also been demonstrated to inhibit SARS-COV-2 replication in vitro and be highly efficacious in murine COVID-19 model assays.

### Materials and Methods

To compare the clinical response and activity of the candidates, the therapeutic response of these candidates against SARS-COV-2 was determined at various multiplicities of infection. Briefly, the SARS-COV-2 virus strain (obtained from CDC BEI Resources Repository) was propagated using Vero E6 cells. Stock virus was prepared after serial passages in Vero E6 cells in infection media. To evaluate the effect of the compounds in vitro, Vero E6 cells were pre-treated with compounds diluted in infection media for 1 hour prior to infection by SARS-CoV-2 virus. Supernatants were collected at various time points (0, 2, 6, 12, 24, 48 and 72 hours) to quantify viral loads by quantitative real-time RT-PCR. The 50% effective concentrations (EC50) of the drug compounds that inhibit viral replication was evaluated.

Cytotoxicity in VeroE6 cells was assessed using the viability assay by the Cell Counting Kit-8 (CCK8) method. Following treatments as mentioned above, cells were treated with 5 mg/mL CCK8 and incubated for 2 hours. The OD value was read at 570 nm on a microplate reader. Cell viability was calculated as the percent ratio of absorbance of the samples against untreated controls.

The novel SARS-Cov-2 in vivo model of the present disclosure was developed using intranasal infections of SARS-COV-2 strain in hACE2 transgenic mice. A vector carrying a human ACE2-coding sequence was introduced into C57BL/6 mice, which subsequently developed a successful hACE2 transgenic mouse strain. When infected with SARS-COV-2 virus, it caused a lethal pulmonary syndrome in mice. The titer of virus was determined by a plaque assay, and mice were anaesthetized and infected intranasally with the indicated dosage of SARS-COV in DMEM. The severity of the infection was adjusted based on the dose amount that was used. Mice infected with low amounts experienced transient weight loss and minor clinical signs of disease. Infection with higher amounts resulted in significant weight loss by day 5, and significant clinical disease, including fever, hunched posture, decreased activity, labored breathing, and subsequently, death.

Treatments were administered at various doses. Negative and positive controls were used to compare therapeutic efficacy and benefit. Mice were weighed and observed for clinical signs daily throughout the study. Clinical changes were noted daily, and biological samples were collected at 0, 1-, 2-, 4-, and 7-days post-infection to allow for profiling of the different phases of infection. The virus load in the lung was quantified at the end of experiment. Morphology and histopathology of the lungs, heart, kidneys, and other organs were conducted and correlated to clinical results. The resolution of clinical symptoms and signs, viral titers, and histopathological parameters were evaluated and compared.

### Results

The anti-PHR agents were screened in a human ACE-2 transgenic COVID-19 mouse model. This model exhibits significant mortality, with approximately 40% mortality at Day 8. The model also exhibits significant morbidity and recapitulates human COVID-19 viral lung pathology, cytokine storm, hemorrhage, fibrosis, labored breathing, lethargy, and wasting disease. Morbidity/PHR can be measured using body weight; a weight loss of approximately 50% is observed in the model by Day 7.

Administration of the anti-PHR agents of the present disclosure completely overcame the mortality rate of the mouse model, with a 100% survival rate observed. In addition, morbidity was drastically reduced; of all of the agents tested, the anti-PHR agents of the present disclosure were the only agents to increase body weight from a baseline level. In addition, a pharmacologically-achievable dose was obtained.

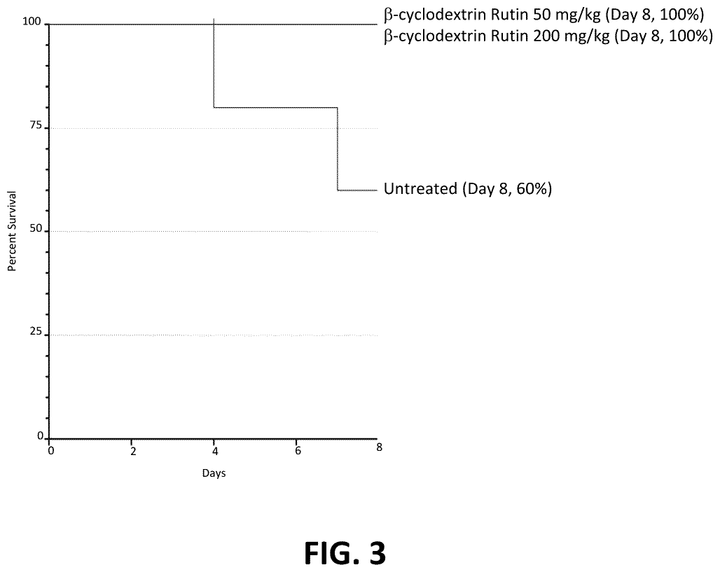

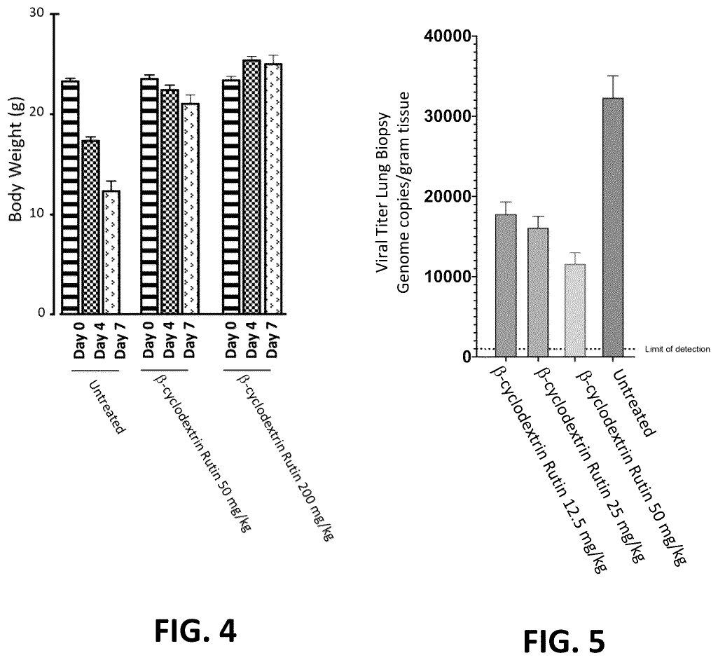

FIGS. 3-5 demonstrate the in vivo efficacy of one anti-PHR agent produced in accordance with the present disclosure (β-cyclodextrin-modified rutin). As can be seen in FIG. 3, a 100% survival rate was observed at 50 mg/kg and 200 mg/kg of the anti-PHR agent. FIG. 4 demonstrates that both 50 mg/kg and 200 mg/kg dosages of the anti-PHR agent decreased morbidity in the mouse model (as measured by body weight), and the 200 mg/kg dose actually increased body weight from a baseline level. FIG. 5 demonstrates that at least moderate anti-viral activity (as determined based on viral titer of a lung biopsy) was observed for the anti-PHR agent and increased at increasing dosage levels.

Next, anti-viral agents were screened for use in the bimodal combinatorial therapy with the anti-PHR agent (β-cyclodextrin-modified rutin). The screening included a broad-based screening in vitro with SARS-COV-2-infected Vero E6 cells, followed by a secondary screening in vivo in the transgenic mouse model described above. Then the anti-viral was chosen based on efficacy, safety, regulatory, cost, and scalability. Anti-viral agents that may be utilized include ivermectin, remdesivir, atorvastatin, montelukast, hydroxychloroquine, chloroquine, catechin, curcumin, quercetin, rutin, nafamostat mesylate, 6-mercaptopurine (6MP), zinc, lopinavir, ritonavir, oseltamivir, fexofenadine, ribavirin, favipiravir, licorice extract, andrographis extract, interferon-beta, dexamethasone, hydrocortisone, methylprednisolone, colchicine, convalescent plasma, ritonavir, molnupiravir, xocova, ensovibep, at least one SARS-COV-2 monoclonal antibody (as described herein above), Tocilizumab, Molnupiravir, vitamin D, zinc, and the like, as well as any combinations thereof.

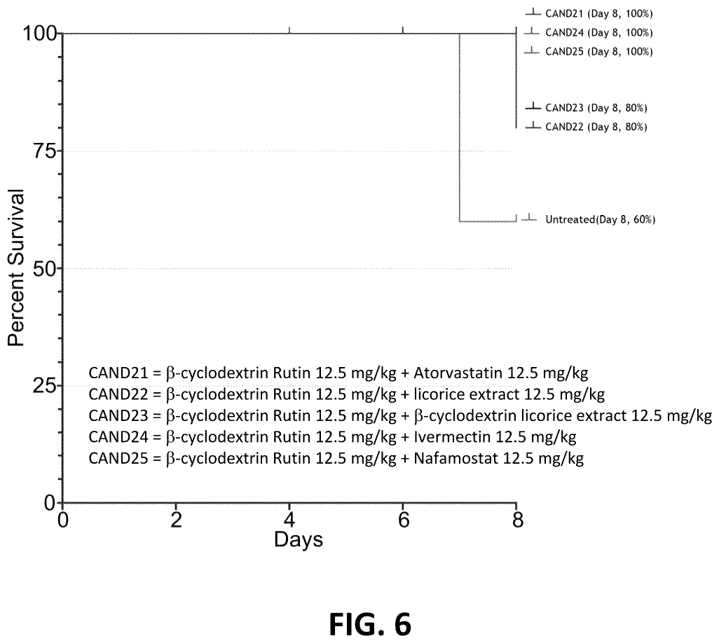

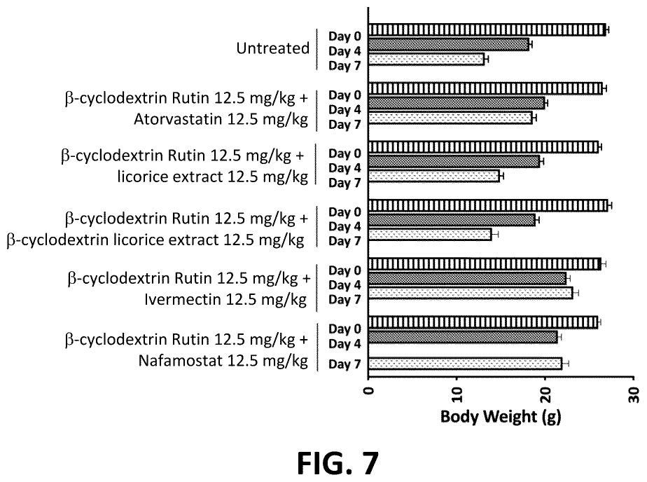

FIGS. 6-7 demonstrate the screening of the efficacy in vivo of combinatorial therapies that include at least one anti-PHR agent (β-cyclodextrin-modified rutin) with various anti-viral agents (i.e., atorvastatin, licorice extract, β-cyclodextrin licorice extract, ivermectin, and nafamostat are shown in FIGS. 6-7; in addition, curcumin, quercetin, rutin, and combinations of two or more anti-viral agents were also tested (data not shown)). As shown in FIG. 6, all of the combinatorial therapies tested were effective in preventing mortality, with a 100% survival rate observed in all instances at Day 6, versus 60% survival in the untreated control arm. Moreover, all treatments increased survival at Day 8 (80%-100% survival) versus the untreated control arm (60% survival). In addition, all of the combinatorial therapies were effective in reducing morbidity to some degree (FIG. 7). The combinatorial therapy tested that was most effective at reducing mortality included ivermectin as the anti-viral agent; this combination is referred to herein as “HBCCT-01.”

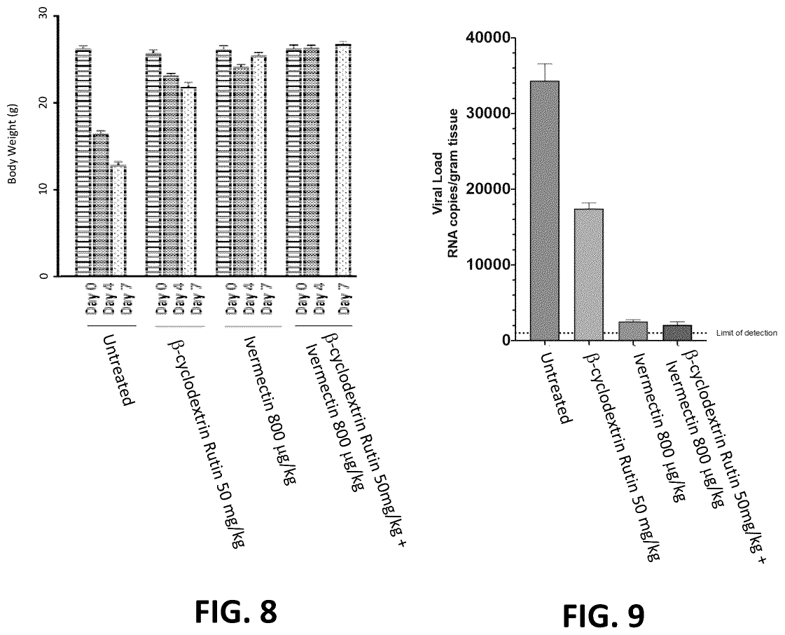

FIGS. 8-9 demonstrate a comparison of the individual components of β-cyclodextrin-rutin and ivermectin to the combinatorial therapy HBCCT-01. As can be seen in FIG. 8, each of β-cyclodextrin-rutin and ivermectin alone decreased morbidity in vivo; however, the combinatorial therapy exhibited a synergistic effect in decreasing morbidity—a slight increase in body weight was even observed. As seen in FIG. 9, β-cyclodextrin-rutin had a limited anti-viral effect relative to the anti-viral agent ivermectin in vivo, and the combinatorial therapy exhibited an even greater anti-viral effect.

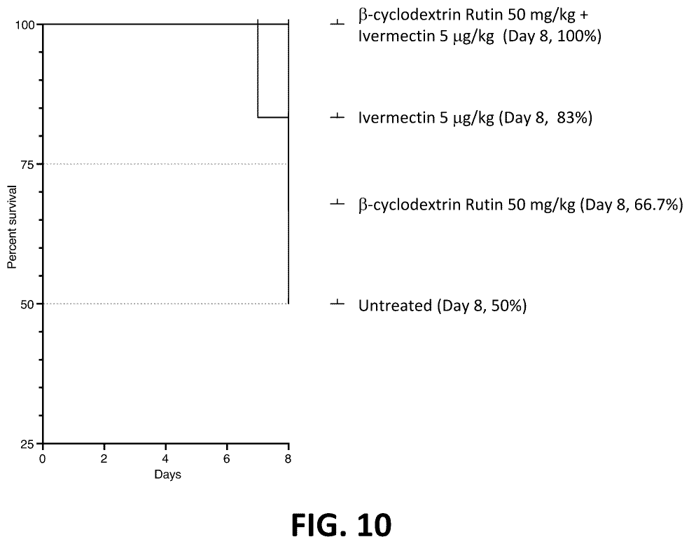

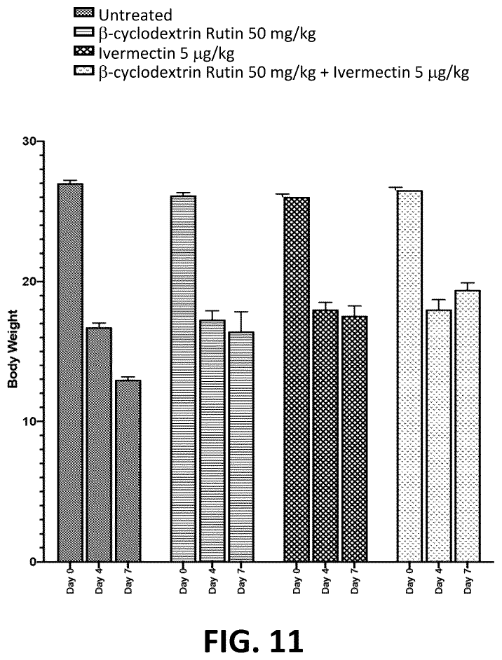

FIGS. 10-11 demonstrate that the combinatorial therapy HBCCT-01 produced a 100% survival rate in vivo at a pharmacologically achievable dose. In addition, these figures also demonstrate the synergistic efficacy of the combinatorial therapy relative to the individual components. These data demonstrate that HBCCT-01 matched the anti-viral activity of the current standard of care but provided a much greater effect on the morbidity by alleviating the signs and symptoms of COVID-19 infection.

### Example 2

Various anti-PHR agents utilized in accordance with the present disclosure could benefit from being modified to increase the bioavailability thereof. As such, the present disclosure also includes methods of modifying anti-PHR agents to provide increased bioavailability when compared to unmodified agent.

The present methods of increasing bioavailability include (for example, but not by way of limitation) the use of a cyclodextrin. Cyclodextrins are a family of cyclic oligosaccharides that possess a hydrophilic outer surface and a lipophilic central cavity or pocket. In the present method, the anti-PHR agent becomes bound, entrapped, and/or complexed within the central pocket of the cyclodextrin, and such binding increases the water solubility and stability of the anti-PHR agent.

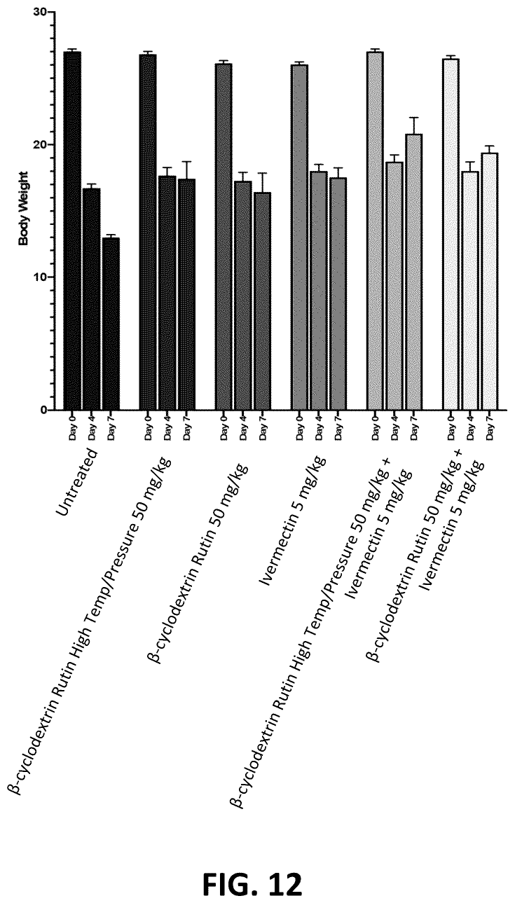

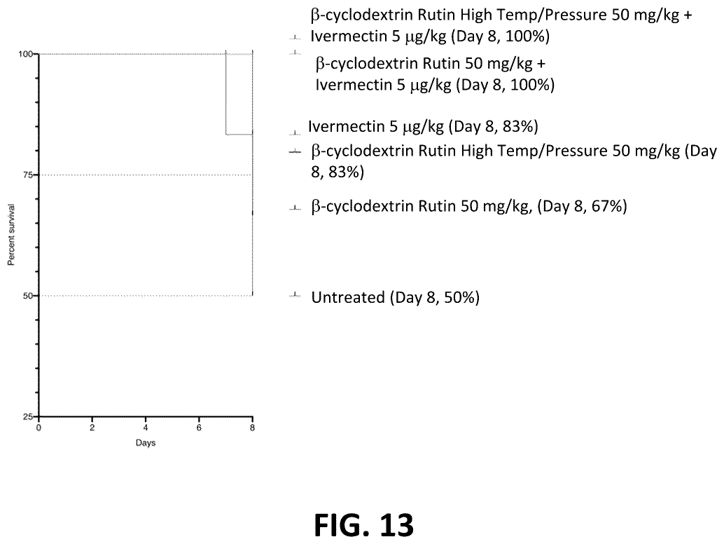

FIGS. 12-13 illustrate the results obtained with the mouse model when using a high bioavailability anti-PHR agent (BCD-rutin) in combination with an anti-viral agent (ivermectin) in the bimodal combinatorial therapy of the present disclosure. The high bioavailability BCD-rutin was prepared by two different ways. The conventional prior art method involves preparing the BCD-rutin complex at room temperature and atmospheric pressure in a kitchen laid mixer (labeled in FIGS. 12-13 simply as “β-cyclodextrin Rutin”). In addition, a novel production method described in U.S. Ser. No. 63/234,806, filed on Aug. 19, 2021, was utilized that involves incubating a cyclodextrin and an agent under conditions that include a temperature above room temperature (such as, but not limited to, a temperature in a range of from about 20° C. to about 300° C.) and a pressure above atmospheric pressure (such as, but not limited to, a pressure in a range of from about 15 PSI to about 200 PSI) for a sufficient amount of time (such as, but not limited to, a period in a range of from about 0.1 hours to about 18 hours) to form the cyclodextrin-modified agent (labeled in FIGS. 12-13 as “β-cyclodextrin Rutin High Temp/Pressure”).

As can be seen in FIG. 12, all of the treatment modalities had some effect on morbidity, with the two bimodal combinatorial therapies showing the greatest effect. Also, the addition of higher temperature and pressure to the production of the high bioavailability anti-PHR agent provided a more robust response on morbidity. The combinatorial therapies both exhibited weight gain at Day 7, with the combinatorial therapy that included BCD-rutin produced under higher temperature and pressure having a greater effect than the therapy that included BCD-rutin produced at room temperature and atmospheric pressure.

FIG. 13 demonstrates that, while a 50% mortality rate was observed in the untreated group, the mortality rate dropped to 17-33% (i.e., 67-83% survival rate) in the single modality treatment groups (i.e., ivermectin alone, BCD-rutin alone, BCD-rutin high temp/pressure alone). However, a 100% survival rate was observed in both of the combinatorial therapy treatment groups.

### Example 3

A clinical trial was conducted using HBCCT-01 (BCD-rutin and ivermectin). The 14-day open-label trial used power and dose ranges based on preclinical results and included four arms, with 10 subjects per arm. The results were compared to the standard of care at the clinic conducting the trial, which included hydroxychloroquine, azithromycin, zinc, and non-steroidal anti-inflammatory drugs.

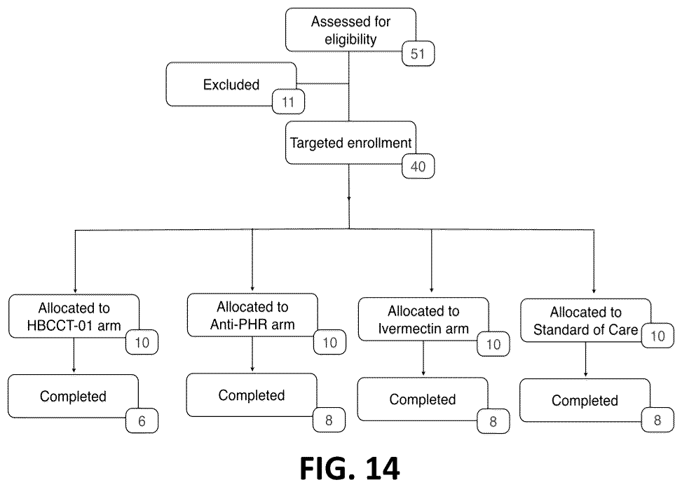

The treatment arms included HBCCT-01 (BCD-rutin at 2 g/day plus a single dose of Ivermectin at 100 μg/kg), anti-PHR (BCD-rutin) alone (2 g/day), Ivermectin alone (100 μg/kg single dose), and standard of care (hydroxychloroquine, azithromycin, and zinc). A primary outcome change was measured by nasopharyngeal viral titer at day 14, based on presence/absence of viral detection by PCR on nasopharyngeal swab. Secondary outcome changes were determined by signs and symptoms, which were collected daily using a custom phone app. Table 1 below illustrates the study parameters that were utilized in the clinical trial, while FIG. 14 contains a flow chart for patient disposition in the clinical trial.

TABLE 1
Study Parameters

| | |
|---|---|
| Endpoints | Time to Defervescence |
| | [Time Frame: baseline to week 2] |
| | Resolution of other COVID-19 symptoms |
| | [Time Frame: baseline to week 2] |
| | Proportion of drug-related adverse events |
| | [Time Frame: baseline to week 2] |
| | Proportion of patients with a negative |
| | SARS-CoV-2 PCR from a nasopharyngeal |
| | swab at day 14 post-treatment |
| | [Time Frame: baseline to week 2] |
| Inclusion Criteria | Patients with mild symptoms of COVID-19 |
| | (cough, fever, anosmia) |
| | Symptoms present for not more than 96 hours |
| | PCR positive for SARS-CoV-2 |
| | Males and females, age 18-65 years old inclusive |
| Exclusion Criteria | Asymptomatic, moderate, severe, or |
| | critically ill patients |
| | COVID-19 pneumonia evidenced by oxygen |
| | saturation <95% or the presence |
| | of lung crackles |
| | Patients with co-morbidities including |
| | diabetes, hypertension, COPD |
| | Patients with pregnancy and on lactation, history |
| | of drug allergies to antiviral medications |

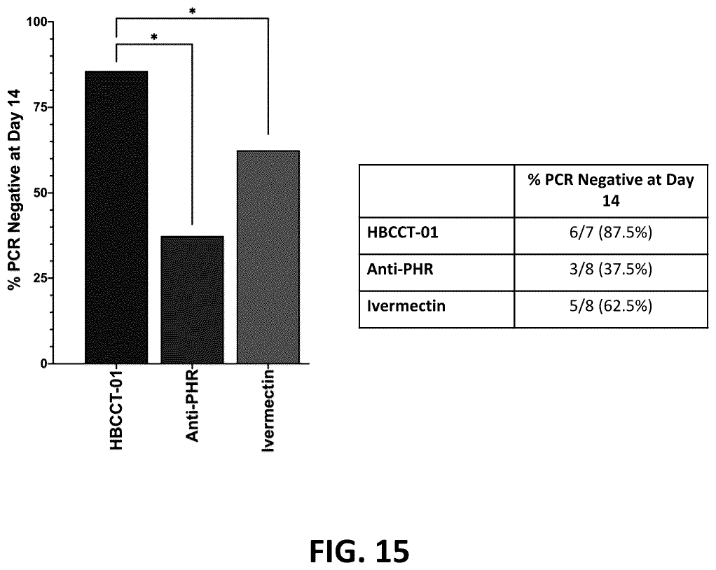

Table 2 and FIG. 15 illustrate the results seen in the clinical trial. As can be seen in Table 2, significant improvements in clinical response rates were seen for the combinatorial therapy HBCCT-01 at both Day 7 and Day 14, when compared to the standard of care and the other treatment arms (i.e., anti-PHR (BCD-rutin) and ivermectin alone). In addition, FIG. 15 demonstrates that a significant increase in the proportion of PCR negative patients was observed in the HBCCT-01 treatment group when compared to the individual components alone (i.e., anti-PHR (BCD-rutin) and ivermectin).

These results demonstrate the efficacy of the bimodal combinatorial therapy of the present disclosure, as well as a vast improvement in ameliorating the signs and symptoms of COVID-19 infection when compared to the current standard of care. In addition, these results demonstrate the synergistic effect observed upon combination of the anti-PHR agent with the anti-viral agent in the bimodal combinatorial therapy of the present disclosure when compared to the individual components alone.

TABLE 2
Outcomes in the Intention-to-Treat Population

| | | | | | Standard |
|---|---|---|---|---|---|
| | | HBCCT-01 | Anti-PHR | Ivermectin | of Care |
| Time to defervescence (days) | | 3 (3 to 4) | 4 (3 to 6) | 4 (3 to 6) | 6 (4 to 8) |
| Time to resolution of COVID | | 4 (3 to 4) | 5 (3 to 7) | 4 (3 to 6) | 8 (6 to 10) |
| symptoms (days) | | | | | |
| Clinical improvement | Day 7 | 10/10 (100%) | 9/10 (90%) | 7/9 (77.7%) | 6/10 (60%) |
| rates (n) | Day 14 | 7/7 (100%) | 7/8 (87.5%) | 8/8 (100%) | 7/8 (87.5%) |

### Example 4

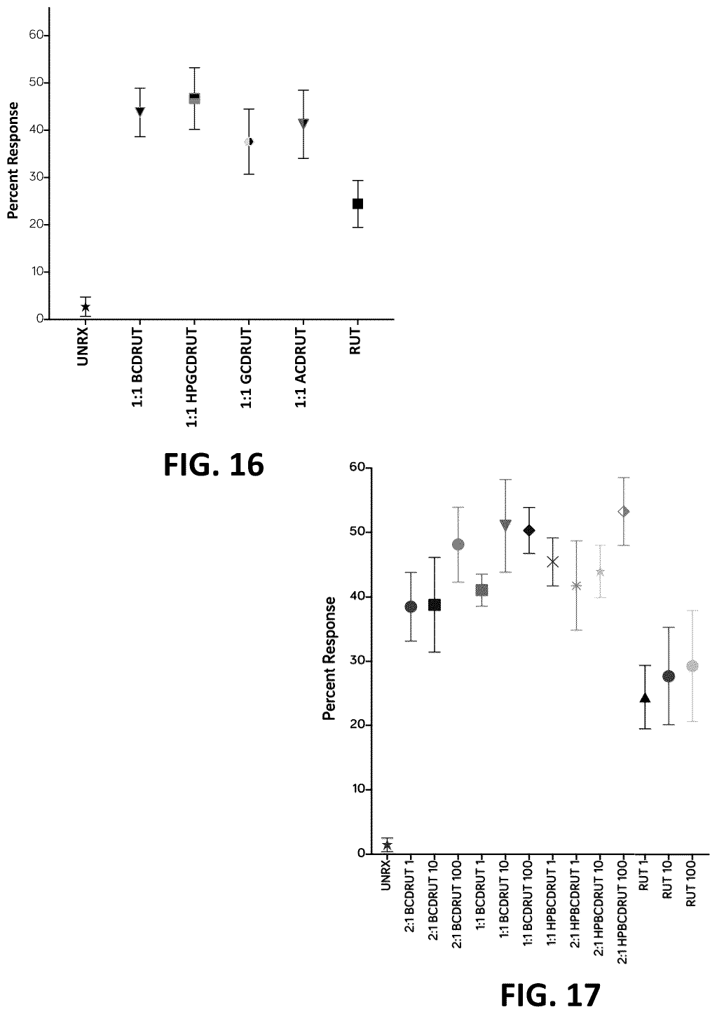

This Example contains an analysis of the anti-PHR agent rutin and various cyclodextrin-modified forms thereof against an in vitro model of SARS-COV-2 described previously. In this Example, rutin was modified with alpha-cyclodextrin (ACDRUT), beta-cyclodextrin (BCDRUT), gamma-cyclodextrin (GCDRUT), Hydroxypropylbetacyclodextrin (HPBCDRUT), and/or Hydroxypropylgammacyclodextrin (HPGCDRUT), and the ability of unmodified rutin and cyclodextrin-modified forms thereof to inhibit SARS-COV-2 replication in vitro was examined. As can be seen in FIG. 16, unmodified rutin inhibited SARS-COV-2 replication by approximately 25% over untreated. However, cyclodextrin modification of rutin resulted in a synergistic increase in inhibition; each of the cyclodextrin-modified rutins ACD-rutin, BCD-rutin, GCD-rutin, and HPGCD-rutin inhibited SARS-COV-2 replication by 40-50% over untreated.

In FIG. 17, additional cyclodextrin-modified rutins were analyzed, including those in which the molar ratio of the cyclodextrin to rutin was varied. As can be seen, unmodified rutin as well as all cyclodextrin-modified rutins tested inhibited SARS-COV-2 replication in vitro at all concentrations tested. In addition, a synergistic effect was observed for all cyclodextrin-modified rutins over unmodified rutin.

### Example 5

In this Example, different combinations of anti-PHR agents were tested in the in vitro model of SARS-COV-2 described previously. A starting formulation containing a combination of various anti-PHR agents was utilized; this combination (labeled as “APHR Combination 1” or “All Supplement”) included Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT). The remaining eight formulations that were analyzed are labeled as SUP2-SUP9 and include all of the agents present in the APHR Combination 1 with the exception of one agent; that is, SUP2 contains all of the APHR Combination 1 except bcd-quercetin, SUP3 contains all of the APHR Combination 1 except hesperidin, etc.

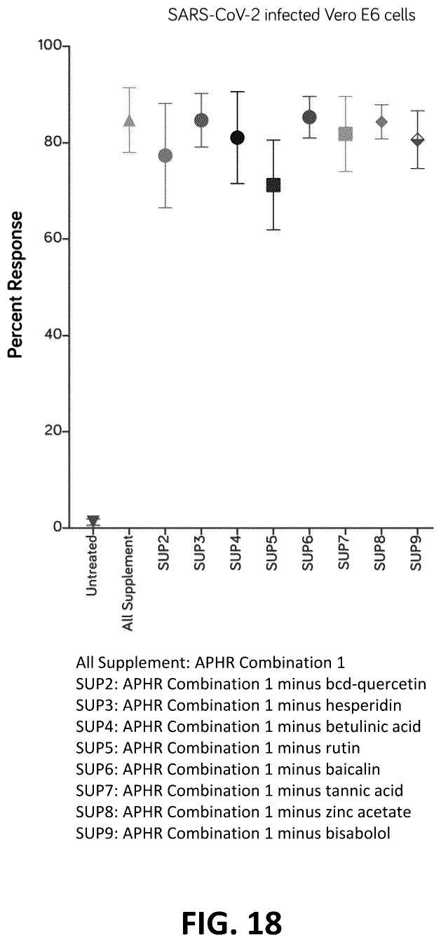

As can be seen in FIG. 18, all nine of the various different formulations of anti-PHR agents greatly inhibited SARS-COV-2 replication by approximately 70-85% over untreated. In addition, removal of single agents from the formulations did not have a drastic effect on this inhibition, thus demonstrating that no single agent is responsible for the activity observed, and that the anti-PHR agents act in concert with one another to provide the drastic effects seen with the combination of agents. Moreover, removal of BCD-quercetin, betulinic acid, rutin, tannic acid, or bisabolol reduced overall activity, demonstrating that these agents have the highest activities in the set.

### Example 6

Table 3 contains a summary of survival data obtained in the in vivo COVID-19 mouse model described previously for various anti-PHR agents alone or in combination with one another (such as, but not limited to, the APHR Combination 1 described above), various anti-viral agents alone, and various combinatorial therapies containing at least one anti-PHR agent and at least one anti-viral agent. As can be seen, a wide variety of anti-PHR agents have been shown to be effective in preventing morbidity and mortality from COVID-19, both as single agents and in combinations of two or more agents.

While the above disclosures describe the inventive concept(s) in conjunction with the specific experimentation, results, and language set forth herein, it is evident that many alternatives, modifications, and variations will be apparent to those skilled in the art. Accordingly, it is intended to embrace all such alternatives, modifications, and variations that fall within the spirit and broad scope of the present disclosure.

TABLE 3
Effects of APHR Agents on Survival in Covid-19 Mouse Model

| Agent | Dose (mg/kg) | Days Treated | Survival (%) |
|---|---|---|---|
| Hydroxypropylbetacyclodextrin Curcumin | 200 | Daily, Pretreatment to Day 7 | 100 |
| Hydroxypropylbetacyclodextrin Curcumin | 66 | Daily, Pretreatment to Day 7 | 100 |
| Quercitin | 200 | Daily, Pretreatment to Day 7 | 100 |
| Quercitin | 66 | Daily, Pretreatment to Day 7 | 80 |
| Hydroxypropylbetacyclodextrin Curcumin + Quercitin | 100 of each | Daily, Pretreatment to Day 7 | 100 |
| APHR Combination 1 | 200 | Daily, Pretreatment to Day 7 | 100 |
| APHR Combination 1 | 50 | Daily, Pretreatment to Day 7 | 80 |
| Betacyclodextrin Rutin | 200 | Daily, Day 1 post-treatment to 7 | 100 |
| Betacyclodextrin Rutin | 50 | Daily, Day 1 post-treatment to 7 | 100 |
| Betacyclodextrin Rutin | 25 | Daily, Day 1 post-treatment to 7 | 80 |
| Betacyclodextrin Rutin + Green tea extract | 25 of each | Daily, Day 1 post-treatment to 7 | 80 |
| Betacyclodextrin Rutin + betacyclodextrin Curcumin | 25 of each | Daily, Day 1 post-treatment to 7 | 100 |
| Betacyclodextrin Rutin + betacyclodextrin Quercitin | 25 of each | Daily, Day 1 post-treatment to 7 | 100 |
| Betacyclodextrin Rutin + Glycyrrhetinic acid | 25 of each | Daily, Day 1 post-treatment to 7 | 100 |
| Hydroxypropylbetacyclodextrin Rutin | 50 | Daily, Day 1 post-treatment to 7 | 100 |
| Hydroxypropylbetacyclodextrin Rutin + | 25 of each | Daily, Day 1 post-treatment to 7 | 100 |
| Hydroxypropylbetacyclodextrin Curcumin | | | |
| Hydroxypropylbetacyclodextrin Rutin + Remdesivir | 25 of each | Daily, Day 1 post-treatment to 7 | 100 |
| Betacyclodextrin Rutin + Hesperidin | 25 of each | Daily, Day 1 post-treatment to 7 | 80 |
| Betacyclodextrin Rutin | 12.5 | Daily, Day 2 post-treatment to 7 | 80 |
| Betacyclodextrin Rutin + Baicalin | 12.5 of each | Daily, Day 2 post-treatment to 7 | 100 |
| Betacyclodextrin Rutin + Rose extract | 12.5 of each | Daily, Day 2 post-treatment to 7 | 100 |
| Betacyclodextrin Rutin + betacyclodextrin Curcumin | 20 and 5 | Daily, Day 2 post-treatment to 7 | 100 |
| Betacyclodextrin Rutin + Atorvastatin | 12.5 of each | Daily, Day 2 post-treatment to 7 | 100 |
| Betacyclodextrin Rutin + Licorice extract | 12.5 of each | Daily, Day 2 post-treatment to 7 | 83 |
| Betacyclodextrin Rutin + Betacyclodextrin Licorice extract | 12.5 of each | Daily, Day 2 post-treatment to 7 | 83 |
| Betacyclodextrin Rutin + Nefamostat mesylate | 12.5 of each | Daily, Day 2 post-treatment to 7 | 83 |
| Remdesivir (positive control) | 200 | Daily, Pretreatment to Day 7 | 100 |
| Remdesivir (positive control) | 50 | Daily, Day 1 post-treatment to 7 | 100 |
| Untreated (negative control) | | | 40-60 (mean = 50) |

1-8. (canceled)

9. A method of treating or reducing the occurrence of a viral infection in a patient, the method comprising the steps of:
(1) administering at least one dose of at least one anti-viral agent to the patient; and
(2) administering at least one dose of at least one anti-Pathogenic Host Response (anti-PHR) agent to the patient, wherein the at least one anti-PHR agent is administered simultaneously or wholly or partially sequentially with the at least one anti-viral agent.

10. The method of claim 9, wherein the viral infection is a coronavirus infection or an influenza infection.

11. The method of claim 10, wherein the coronavirus infection is selected from the group consisting of include severe acute respiratory syndrome coronavirus (SARS-COV), severe acute respiratory syndrome coronavirus 2 (SARS-COV-2), Middle East respiratory syndrome coronavirus (MERS-COV), human coronavirus 229E (HCoV-229E), human coronavirus OC43 (HCoV-OC43), human coronavirus NL63 (HCoV-NL63), and human coronavirus HKU1 (HCoV-HKU1), or a variant thereof.

12. The method of claim 11, wherein the coronavirus is SARS-COV-2 or a variant thereof.

13. The method of claim 9, wherein the at least one anti-viral agent is selected from the group consisting of ivermectin, remdesivir, atorvastatin, montelukast, hydroxychloroquine, chloroquine, catechin, curcumin, quercetin, rutin, nafamostat mesylate, 6-mercaptopurine (6MP), zinc, lopinavir, ritonavir, oseltamivir, fexofenadine, ribavirin, favipiravir, licorice extract, andrographis extract, interferon-beta, dexamethasone, hydrocortisone, methylprednisolone, colchicine, convalescent plasma, ritonavir, molnupiravir, xocova, ensovibep, at least one SARS-COV-2 monoclonal antibody, Tocilizumab, Molnupiravir, vitamin D, zinc, and combinations thereof.

14. The method of claim 9, wherein the at least one anti-PHR agent is selected from the group consisting of rutin, sodium rutin, curcumin, quercetin, hesperidin, baicalin, green tea extract, rose extract, betulinic acid, tannic acid, bisabolol, a modified form thereof, and combinations thereof.

15. (canceled)

16. The method of claim 15, wherein the at least one anti-PHR agent is a cyclodextrin-modified form of at least one of rutin, sodium rutin, curcumin, and quercetin, and wherein the cyclodextrin is selected from the group consisting of alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), or hydroxypropyl gamma-cyclodextrin (HPGD).

17. The method of claim 9, wherein the at least one anti-viral agent comprises ivermectin, and the at least one anti-PHR agent comprises at least one of rutin, sodium rutin, BCD-rutin, or HPBCD-rutin.

18. The method of claim 9, wherein the at least one anti-PHR agent comprises at least one of curcumin, BCD-curcumin, or HPBCD-curcumin.

19. The method of claim 9, further comprising the step of:
(3) administering at least one additional dose of the at least one anti-PHR agent to the patient.

20. The method of claim 19, wherein step (3) is repeated on a daily basis for a period in a range of from about 1 day to about 60 days.

21. The method of claim 9, wherein the dose of step (1) is in a range of from about 1 μg/kg to about 100 mg/kg, and wherein the dose of step (2) is in a range of from about 1 μg/kg to about 500 mg/kg.

22-32. (canceled)

33. A kit for treatment of COVID-19 infection, comprising:
at least one anti-viral agent; and
at least one anti-PHR agent.

34. The kit of claim 33, wherein:
the at least one anti-viral agent is selected from the group consisting of ivermectin, remdesivir, atorvastatin, montelukast, hydroxychloroquine, chloroquine, catechin, curcumin, quercetin, rutin, nafamostat mesylate, 6-mercaptopurine (6MP), zinc, lopinavir, ritonavir, oseltamivir, fexofenadine, ribavirin, favipiravir, licorice extract, andrographis extract, interferon-beta, dexamethasone, hydrocortisone, methylprednisolone, colchicine, convalescent plasma, ritonavir, molnupiravir, xocova, ensovibep, at least one SARS-COV-2 monoclonal antibody, Tocilizumab, Molnupiravir, vitamin D, zinc, and combinations thereof; and
wherein the at least one anti-PHR agent is selected from the group consisting of rutin, sodium rutin, curcumin, quercetin, hesperidin, baicalin, green tea extract, rose extract, betulinic acid, tannic acid, bisabolol, a modified form thereof, and combinations thereof.

35. (canceled)

36. The method of claim 35, wherein the at least one anti-PHR agent is a cyclodextrin-modified form of at least one of rutin, sodium rutin, curcumin, and quercetin, and wherein the cyclodextrin is selected from the group consisting of alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), or hydroxypropyl gamma-cyclodextrin (HPGD).

37. (canceled)

38. A pharmaceutical composition, comprising:
at least one anti-viral agent;
at least one anti-PHR agent; and
at least one pharmaceutically acceptable carrier in which the at least one anti-viral agent and at least one anti-PHR agent are disposed.

39. The pharmaceutical composition of claim 38, wherein:
the at least one anti-viral agent is selected from the group consisting of ivermectin, remdesivir, atorvastatin, montelukast, hydroxychloroquine, chloroquine, catechin, curcumin, quercetin, rutin, nafamostat mesylate, 6-mercaptopurine (6MP), zinc, lopinavir, ritonavir, oseltamivir, fexofenadine, ribavirin, favipiravir, licorice extract, andrographis extract, interferon-beta, dexamethasone, hydrocortisone, methylprednisolone, colchicine, convalescent plasma, ritonavir, molnupiravir, xocova, ensovibep, at least one SARS-COV-2 monoclonal antibody, Tocilizumab, Molnupiravir, vitamin D, zinc, and combinations thereof; and
wherein the at least one anti-PHR agent is selected from the group consisting of rutin, sodium rutin, curcumin, quercetin, hesperidin, baicalin, green tea extract, rose extract, betulinic acid, tannic acid, bisabolol, a modified form thereof, and combinations thereof.

40. (canceled)

41. The pharmaceutical composition of claim 40, wherein the at least one anti-PHR agent is a cyclodextrin-modified form of at least one of rutin, sodium rutin, curcumin, and quercetin, and wherein the cyclodextrin is selected from the group consisting of alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), or hydroxypropyl gamma-cyclodextrin (HPGD).

42. The pharmaceutical composition of claim 38, wherein the at least one anti-viral agent comprises ivermectin, and wherein the at least one anti-PHR agent comprises at least one of rutin, sodium rutin, BCD-rutin, or HPBCD-rutin.

43. The pharmaceutical composition of claim 38, further defined as comprising at least four of Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT).

44-67. (canceled)
